(19) United States
(12) Patent Application Publication
(10) Pub. No.: US 2025/0032199 A1
(43) Pub. Date: Jan. 30, 2025

(54) MICROPHONE DIRECTIONALITY CONTROL BASED ON SURGEON'S COMMAND

(71) Applicant: Digital Surgery Systems, Inc., Goleta, CA (US)

(72) Inventors: Saurabh Kotian, Goleta, CA (US); Alan Fridman, Goleta, CA (US); Stephen Minne, Goleta, CA (US)

(21) Appl. No.: 18/837,414

(22) PCT Filed: Feb. 8, 2023
(86) PCT No.: PCT/US2023/062228

§ 371 Date: Aug. 9, 2024

Related U.S. Application Data
(60) Provisional application No. 63/308,659, filed on Feb. 10, 2022.

(51) Int. Cl. A61B 34/00; A61B 17/00; A61B 34/30; A61B 90/00; A61B 90/25

(52) U.S. Cl. CPC A61B 34/25; A61B 17/00; A61B 34/30; A61B 90/06; A61B 90/25; A61B 2017/00203; A61B 2090/067

(57) ABSTRACT

New and innovative systems and methods are described for providing microphone directionality based on a surgeon's command, for use in surgical environmnents. An example method may include: receiving, via a respective sensor for each of one or more rotating elements of each of one or more robotic arms connecting a digital surgical microscope to the computing device, an angle information for each rotating element; determining, based on the angle information for each rotating element, a joint angle information for the digital surgical microscope; determining, based on the joint angle information, a location of a head of the digital surgical microscope respective to a microphone device; and activating, based on the location, a first channel of a plurality of channels of the microphone device.

### PRIORITY CLAIM

The present application claims priority to and the benefit of U.S. Provisional Patent Application 63/308,659, filed Feb. 9, 2022, the entirety of which is incorporated herein by reference.

### TECHNICAL FIELD

Certain aspects of the present disclosure generally relate to surgical systems, and specifically relate to microphone controls for surgical systems.

### BACKGROUND

Surgeons often rely on their hands to achieve various objectives during surgery. For example, a surgeon may use their hands for moving the microscope, accessing surgical tools, and performing a surgical procedure in a patient. A surgeon may often need to interrupt performance of their surgical procedure to adjust or control the surgical visualization system. For example, a surgeon may need to adjust the focus, zoom, and/or intensity of a microscope during a surgery. Such interruptions may often impact the performance of a surgery, by causing delays, diverting a surgeon's attention, or otherwise increasing the risk of mistakes. One way for a surgeon to mitigate the impact of these interruptions is to allow the surgeon to control various aspects of a surgical visualization system via a voice command.

Existing voice control systems can provide non-movement microscope control (i.e., zoom, focus, autofocus, and white light), image and color controls (i.e., next image and previous image modes), media controls (i.e., snapshot control, stop and start recording modes), and hyperspectral controls (i.e., DIR 800 on/off, light control, and playback, and DUV 400 on/off and light control). However, existing voice control system suffer from performance problems with respect to response time and compute load (often requiring network access) as well as in accuracy and miss rate (e.g., “precision” and “recall”).

Various embodiments of the present disclosure address one or more shortcomings of surgical visualization and voice control systems described above.

### SUMMARY

The present disclosure provides new and innovative systems and methods for providing microphone directionality based on a surgeon's command, for use in surgical environments. In an example, a system is disclosed for providing microphone directionality based on a surgeon's command. The system may include, e.g., a movable cart; a mast affixed to the movable cart; one or more robotic arms; a coupler affixed to a distal end of a robotic arm of the one or more robotic arms; a digital surgical microscope affixed to the coupler; and a microphone device affixed to the mast. Each robotic arm may be associated with one or more rotating elements. Each rotating element may be associated with a respective sensor to detect angle information. The microphone device may include a plurality of channels associated with a plurality of directions (e.g., a first channel associated with a first direction, a second channel associated with a second direction, etc.).

The system may also include a processor, and memory storing computer-executable instructions. When executed by the processor, the memory may cause the system to: receive, via the respective sensor for each of the one or more rotating elements, an angle information for each rotating element; determine, based on the angle information for each rotating element, a joint angle information for the digital surgical microscope; determine, based on the joint angle information, a location of a head of the digital surgical microscope respective to the microphone device; and activate, based on the location, a first channel of the plurality of channels associated with the plurality of directions, wherein first channel is associated with a first direction of the plurality of directions indicative of the location. In one embodiment, the instructions may further cause the system to deactivate, based on the location, remaining channels of the plurality of channels associated with the plurality of directions.

In one embodiment, the location of the head of the digital surgical microscope may be determined by: determining, upon an initialization of the system, an initial location of the microscope; activating, based on the initial location, an initial channel of the plurality of channels; and generating, based on a comparison of the initial location to the location of the microscope, a distance vector of the microscope. For example, prior to activating the first channel, the processor may: determine that the distance vector of the microscope satisfies a predetermined threshold; and deactivate the initial channel of the plurality of channels. The first channel may be different from the initial channel. Also or alternatively, the processor may determine that the distance vector of the microscope does not satisfy a predetermined threshold. In such embodiments, the initial channel may comprise the first channel, and activating the first channel may comprise maintaining the activation of the initial channel. In some embodiments, the instructions, when executed, may cause the system to: determine, based on the location of the head of the digital surgical microscope, a proximity of the digital surgical microscope to the microphone device; and adjust, based on the proximity, a gain of the microphone device.

In one embodiment, the instructions, when executed, may cause the system to filter, via a low pass and high pass filter, an audio signal received from the first channel to increase a signal to noise ratio (SNR). Furthermore, one or more equalizers of the microphone device may be modified to isolate a voice command associated with the audio signal.

In one embodiment, the microphone device may further comprise an output module for converting voice commands filtered through one or more of the plurality of channels into digital signals. The instructions, when executed, may cause the system to: receive, via the output module of the microphone device, a digital signal corresponding to a voice command. The voice command may be identified, based on the digital signal, as a microscope movement command. The microscope movement command may comprise, for example, one or more of: an X-Y movement command along a field of view; a lock-to-target command within the field of view; a Z-axis movement command towards or away from the field of view; or a yaw movement command. The instructions, when executed, may thus cause the system to execute the microscope movement command.

In some embodiments, the system (e.g., via its processor) may identify, based on the digital signal, the voice command as one of: a focus command, an autofocus command, a zoom command, a white light intensity command, a toggle command, a snapshot command, an image and/or color adjustment command; an image scrolling command; a recording command; a bookmark command; or a hyperspectral imaging command. In some aspects, the hyperspectral imaging command may comprise, for example, one or more of: the hyperspectral imaging command is one or more of: a toggle command for near infrared (NIR) imaging (e.g., at the 800 nanometer visible region (NIR 800)); a playback command for the NIR imaging; a light control command for the NIR imaging; a toggle command for near ultraviolent (NUV) imaging (e.g., at the 400 nanometer visible region (NUV 400)); a playback command for the NUV imaging; a light control command for the NUV imaging; a toggle command for fluorescence imaging; a playback command for the fluorescence imaging; a light control command for the fluorescence imaging; a toggle command for processing-in-pixel (PIP); or a swap sources command. The system may thus cause the digital surgical microscope to execute the voice command.

In an example, a method of controlling microphone directionality in a surgical environment is disclosed. The method may be performed by a processor associated with a computing device. The method may include: receiving, via a respective sensor for each of one or more rotating elements of each of one or more robotic arms connecting a digital surgical microscope to the computing device, an angle information for each rotating element; determining, based on the angle information for each rotating element, a joint angle information for the digital surgical microscope; determining, based on the joint angle information, a location of a head of the digital surgical microscope respective to a microphone device; and activating, based on the location, a first channel of a plurality of channels of the microphone device. The plurality of channels of the microphone device may be associated with a respective plurality of directions, such that the first channel is associated with a first direction of the plurality of directions. The method my further comprise deactivating, based on the location and responsive to the activation of the first channel, remaining channels of the plurality of channels of the microphone device.

In some embodiments, the method may further comprise: determining, upon an initialization of the system, an initial location of the microscope; activating, based on the initial location, an initial channel of the plurality of channels; and generating, based on a comparison of the initial location to the location of the microscope, a distance vector of the microscope. In some aspects, determining the location of the head of the digital surgical microscope comprises: prior to activating the first channel, determining that the distance vector of the microscope satisfies a predetermined threshold; and deactivating the initial channel of the plurality of channels, wherein the first channel is different from the initial channel. Also or alternatively, determining the location of the head of the digital surgical microscope comprises: determining that the distance vector of the microscope does not satisfy a predetermined threshold, wherein the initial channel comprises the first channel, wherein the activating the first channel comprises maintaining the activation of the initial channel.

In some embodiments, the method may further comprise: determining, based on the location of the head of the digital surgical microscope, a proximity of the digital surgical microscope to the microphone device; and adjusting, based on the proximity, a gain of the microphone device.

In some embodiments, the method may further comprise: filtering, via a low pass and high pass filter, an audio signal received from the first channel to increase a signal to noise ratio (SNR); and modifying one or more equalizers of the microphone device to isolate a voice command associated with the audio signal.

In some embodiments, the method may further comprise: receiving, via the microphone device, a digital signal corresponding to a voice command; identifying, based on the digital signal, the voice command as a microscope movement command; and causing the digital surgical microscope to execute the microscope movement command. For example, the microscope movement command may include but are not limited to one or more of: an X-Y movement command along a field of view; a lock-to-target command within the field of view; a Z-axis movement command towards or away from the field of view; or a yaw movement command.

In some embodiments, the method may further comprise: receiving, via the microphone device, a digital signal corresponding to a voice command; identifying, based on the digital signal, the voice command as one of: a focus command; an autofocus command; a zoom command; a white light intensity command; a toggle command; a snapshot command; an image and/or color adjustment command; an image scrolling command; a recording command; a bookmark command; or a hyperspectral imaging command; and causing the digital surgical microscope to execute the voice command. In some aspects, the hyperspectral imaging command may include but is not limited to one or more of: a toggle command for near infrared imaging; a playback command for the NIR imaging; a light control command for the NIR imaging; a toggle command for near ultraviolent (NUV) imaging; a playback command for the NUV imaging; a light control command for the NUV imaging; a toggle command for fluorescence imaging; a playback command for the fluorescence imaging; a light control command for the fluorescence imaging; a toggle command for processing-in-pixel (PIP); or a swap sources command.

In an example, a non-transitory computer-readable medium for use on a computer system is disclosed. The non-transitory computer-readable medium may contain computer-executable programming instructions may cause processors to perform one or more steps or methods described herein.

### BRIEF DESCRIPTION OF THE FIGURES

FIG. 1 shows a diagram of a surgical environment including a microphone device having a plurality of channels corresponding to a plurality of directions, according to an example embodiment of the present disclosure.

FIG. 2 shows a flow diagram of a method of controlling microphone directionality in a surgical environment, according to an example embodiment of the present disclosure.

FIG. 3 shows a screenshot of a user interface associated with a system for controlling microphone directionality in a surgical environment, according to an example embodiment of the present disclosure.

FIGS. 4-7 show illustrations, in different rotational positions, of a surgical environment for providing microphone directionality based on a surgeon's command, according to example embodiments of the present disclosure.

FIG. 8 is an illustration showing a surgical environment for providing microphone directionality based on a surgeon's command, in which a microphone device is placed in multiple locations to test its efficacy, in accordance with non-limiting embodiments of the present disclosure.

FIG. 9 is an illustration of the location of different users in a surgical environment for testing the optimal location of the microphone device, in accordance with a non-limiting embodiment of the present disclosure.

FIG. 10 is a graph of a mean sound analysis of a disc microphone device placed in different locations and orientations in a surgical environment, in accordance with a non-limiting embodiment of the present disclosure.

FIG. 11 is a graph of a mean sound analysis of a microphone device placed at the digital surgical microscope head in a surgical environment, in accordance with a non-limiting embodiment of the present disclosure.

FIG. 12 is a graph of a comparison of the mean sound analysis of a disc microphone device placed at a robotic mast with a sound analysis of a microphone device placed at a digital surgical microscope head in a surgical environment, in accordance with a non-limiting embodiment of the present disclosure.

FIG. 13 is a graph of a maximum sound analysis of a disc microphone device placed in different locations and orientations in a surgical environment, in accordance with a non-limiting embodiment of the present disclosure.

FIG. 14 is a graph of a maximum sound analysis of a microphone device placed at the digital surgical microscope head in a surgical environment, in accordance with a non-limiting embodiment of the present disclosure.

FIG. 15 is a graph of a comparison of the maximum sound analysis of a disc microphone device placed at a robotic mast with a sound analysis of a microphone device placed at a digital surgical microscope head in a surgical environment, in accordance with a non-limiting embodiment of the present disclosure.

FIG. 16 shows a traditional (e.g., shotgun) microphone mounted on motorized turrets and implementing systems and methods for providing microphone directionality based on surgeon command, in accordance with a non-limiting embodiment of the present disclosure

### DETAILED DESCRIPTION

As previously discussed, there is a need for providing control of surgical visualization and navigation systems to the surgeon through non-manual means (e.g., voice control) so that the surgeon can use their hands to perform the surgery without interruption, loss of time and focus. However, existing voice control systems suffer from performance problems with respect to response time, compute load, accuracy, and miss rate. The present disclosure relates in general to a microphone-based voice control system in a surgical environment. Specifically, the microphone device is rendered more receptive to a surgeon's commands by determining the location of the digital surgical microscope, which is understood to be the likely location of the surgeon during surgery. Based on this determination of the location, one or more direction-based channels of the microphone device may be activated, and the remaining direction-based channels may be deactivated, e.g., to optimize the sound signals coming from the direction where the surgeon is located.

Various embodiments of the present disclosure may include voice control as a feature to control certain functionalities of surgical visualization and navigation systems not including robotic movements. Voice control may provide an added benefit to the surgeons by providing them a completely hands-free surgical environment. For example, surgeons may be able to control optical functionalities such as focus, zoom, white light intensity, toggle infrared light functionalities (e.g., DIR 800), or toggle ultraviolet light functionalities (e.g., DUV 400), for example, using voice commands. Surgeons may also be able to take snapshots, start/stop screen recording and control other similar functionalities using voice commands.

In some embodiments, microphone hardware may be configured, modified, and/or reengineered to perform one or more functions described herein. For example, various embodiments of the present disclosure may utilize disc microphone devices having direction-based channels (e.g., the SHURE MXA310). Such disc microphone devices may provide, in their default configuration, uniform radial pickup pattern in all direction-based channels. However, one or more methods described herein may allow one or more of the direction-based channels to remain or be activated to retain pickup, while remaining direction-based channels may be deactivated. For example, the direction, gain, and/or channels of such microphone devices may be controllable via software API's.

The present disclosure may provide various benefits to the surgeon and medical personnel, and improve surgery experience and outcome. For example, integrating the direction of the microphone with the location of the digital surgical microscope (DSM) (e.g., as determined via robotic arms leading to the DSM) may provide the user (e.g., surgeon) with the ability to control the system via voice commands. This capability may free the user from the burden of doing any additional setup concerning the position and orientation of the cart with respect to the user's position. The centering of the direction of the microphone towards the surgeon (DSM head) via the activation (and deactivation) of direction-based channels may improve (e.g., maximize) gain and reduce background noise. For example, the deactivation (e.g., turning off) of receiver channels that are away from the surgeon (as deduced from the position of the DSM head) may reduce background noise significantly. Furthermore, the surgeon need not have to wear any additional equipment to provide voice commands to the system.

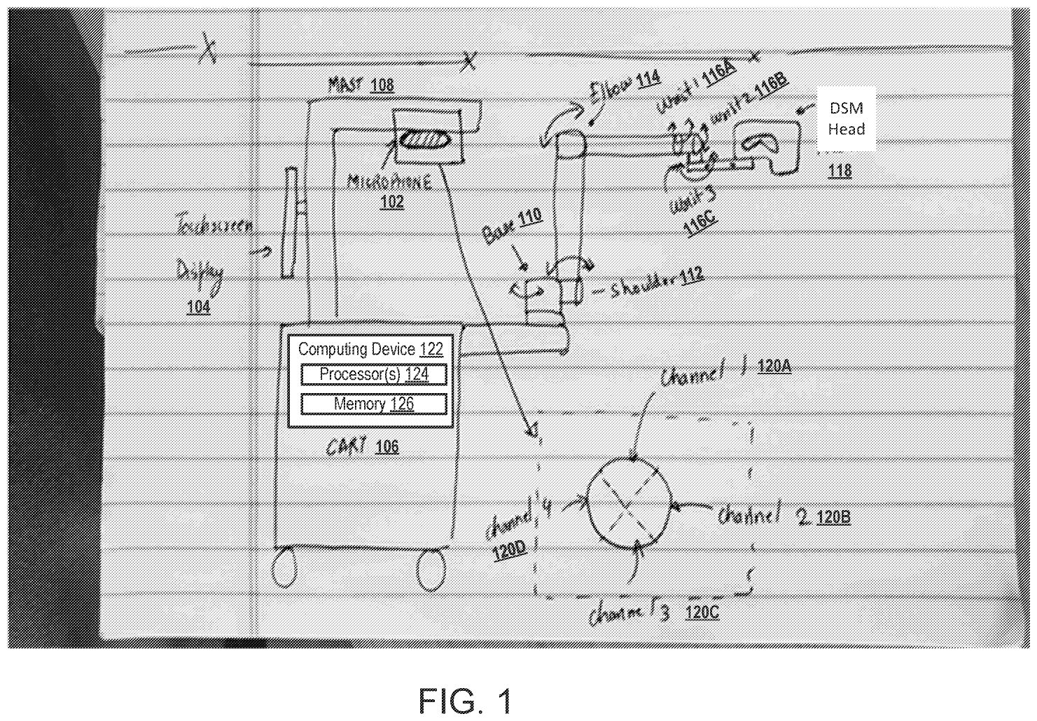

FIG. 1 shows a diagram of a surgical environment including a microphone device having a plurality of channels corresponding to a plurality of directions, according to an example embodiment of the present disclosure. As shown in FIG. 1, the surgical environment 100 may include a microphone device 102 attached to a mast 108 of a cart 106 that carries, or renders portable, the surgical visualization and navigation system. A touch screen display 104 may allow medical personnel (not shown) to see the field of view of a digital surgical microscope (DSM) camera (e.g., the field of view of the DSM Head 118). The DSM head 118 may be coupled to the rest of the system via a plurality of robotic arms and rotating elements. For example, as shown in FIG. 1, in the order of increasing proximity to the DSM head 118, the cart may be coupled to a base 110, a shoulder 112, an elbow 114, waist 1 116A, waist 2 116B, and waist 3 116C, each of which may be rotatable along the arrows shown. Based on the use case, type of surgery, patient and surgeon positioning, the cart 106 can be placed around the patient table at different locations and orientations making it challenging to direct the microphone input channel towards the location of the surgeon. Thus, various embodiments of the present disclosure describe systems and methods for controlling microphone directionality in order to effectively capture surgeon's voice commands.

In some embodiments, the microphone device 102 may be disc shaped and may be attached to the mast 108 horizontally (e.g., such that the disc is parallel to the floor). The microphone device 102 may have a plurality of direction based channels as shown (e.g., channel 1 120A, channel 2, 120B, channel 3 120C, channel 4, 120D). These channels may each be associated with a specific direction e.g., so that a given channel may be most capable or effective at receiving sound signals coming from their respective direction. While four channels are shown for the microphone, the microphone may include fewer channels, such as three or two channels, or more channels (e.g., as many as five, six, eight, or ten channels). The directionality of the channels may be equal, such that, for example, three channels may have 120 degrees of directionality. In other embodiments, the directionality of the channels may vary. In one embodiment, the disc may be rotatable, such that the channels may be better poised to receive sound signals from certain directions. The microphone device 102 may be keyed to ensure that the center of a certain channel (e.g., channel 1 120A) of the plurality of channels is closely aligned towards a base joint (e.g., base 110) of the plurality of robotic arms and rotating elements leading to the DSM head. This can be achieved by using the dimensions of the cart 106 to calculate a direction vector to the robot base joint (e.g., base 110).

In some embodiments, the cart 106 may comprise a computing device 122 having memory 126 and one or more processors 124. The memory 126 may store computer executable instructions that, when executed by the one or more processors 124, may cause the computing device 122 to perform one or more steps or methods described herein. Also or alternatively, the microphone device 102 may comprise the computing device, and/or may include the one or more processors and memory.

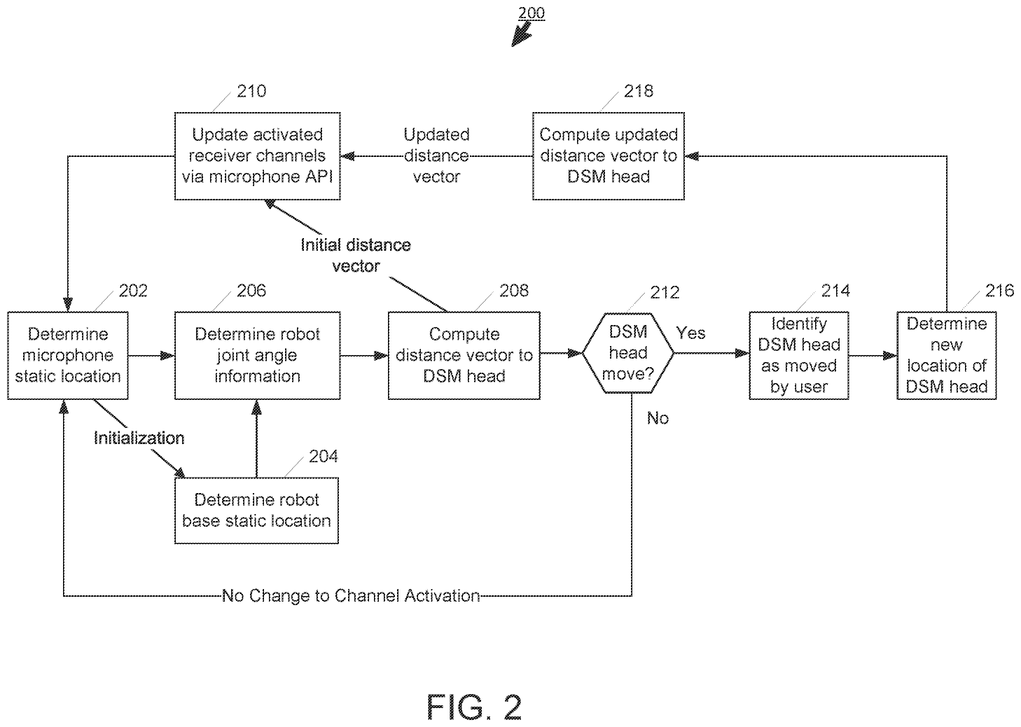

FIG. 2 shows a flow diagram of a method of controlling microphone directionality in a surgical environment, according to an example embodiment of the present disclosure. The method may be performed by a processor (e.g., one or more processors 124 of the computing device 122 and/or of the microphone device 102). As previously discussed in relation to FIG. 1, the microphone device may be statically mounted on a mast (e.g., mast 108) of a surgical navigation and/or visualization system, and the microphone device may be a circular disc containing a plurality of channels. The microphone device may be keyed to ensure that the center of a first channel of the plurality of channels is closely aligned towards a base joint (e.g., base 110) of the plurality of robotic arms and rotating elements leading to the DSM head. As shown in FIG. 2, the flow diagram may describe functionalities associated with initializing the microphone's active channels based on an initial microscope position and updating the microphone's active channels based on an updated microscope position.

Method 200 may begin with an initialization process of the microphone voice control system. For example, as part of the initialization, the processor may determine the static location of the microphone (block 202). In at least one embodiment, the static location may be designated as an origin point in a Cartesian coordinate system. In some aspects, the static location of the microphone may be inputted by a user. The initialization may continue with the processor determining the static location of the robot base joint (e.g., base 110) (block 204). In some aspects, the static location of the robot base joint may be the distance spanning the first extending robotic arm jutting out from the cart 106 of the surgical visualization and navigation system. The static location of the robot base joint may be hardwired or inputted into the memory 126 associated with the computing device 122 and/or microphone device 102. In some aspects, the static locations of other robotic arms (e.g., shoulder 112, elbow 114, waist 1 116, waist 2 116B, and waist 3 116C) may also be determined, as part of the initialization process.

Furthermore, the processor may determine a robotic joint angle information (block 206). In some aspects, the robotic joint angle information may be determined by determining the angles of each of the robotic joints interspersed between the robotic arms leading from the cart 106 to the DSM head 118 (block 204). For example, the processor may receive angle information from sensors detecting angles of the robotic joints associated with base 110, shoulder 112, elbow 114, waist 1 116A, waist 2 116B, and waist 3 116C.

Upon initialization, the system calculates the initial microscope position (storage position) by computing the static distance vector to the DSM head (block 208). The static distance vector may be computed, for example, by summing a series of vectors formed by the joint angle information and the length of the robotic arms leading to the DSM head (e.g., the static direction vector to the robot base joint).

The initial distance vector to the DSM head may be used to update activated receiver channels of the microphone device (block 210). For example, the distance vector information may be used to activate (e.g., turn ON) the microphone channel(s) that are closest to the DSM head via the API. In some aspects, the remaining microphone channels, which are not as close to the DSM head may be (e.g., turned OFF). The various microphone channels may be activated and/or deactivated via a microphone application programming interface (API). In some aspects, the computing device 122 may communicate with the microphone API based on the one or more steps performed, and information received by, the computing device 122.

After initialization, the processor may periodically monitor changes in the position of the DSM head (block 212: “DSM Head move?”). The changes may be determined by analyzing any changes to any of the robot joint angles. For example, the computing device may periodically receive sensor readings of angle measurements from each of the robotic joints, and identify, for each robotic joint, whether a delta from a previous reading of an angle of the respective robotic joint exceeds a predetermined threshold. If the computing device determines that there are no changes to the position of the DSM Head (e.g., DSM head move? No), the computing device may repeat steps 202 through 208, e.g., as part of monitoring the DSM head position. No changes may be made to the activation or deactivation of the microphone channels.

If there is a change in the position of the DSM head (e.g., DSM head move? Yes), the computing device may identify the DSM head as having been moved by the user (block 214). The computing device may determine a new location of the DSM head (block 216). For example, the computing device may determine a set of new robotic joint angles of the robotic joints leading to the new position of the DSM head. An updated distance vector to the DSM head may be computed (block 218). In at least one embodiment, individual vectors based on the length of individual robotic arms leading to the DSM head and updated angles of the individual robotic arms may be summated to determine the updated distance vector to the DSM head. Thus, any movement done by the user to the DSM may thereafter be captured as a change to the DSM head, and the distance vector from the microphone device to the DSM head may be re-calculated, e.g., based on updated joint angles.

The updated distance vector may be used to update activated receiver channels via the microphone API (block 210). For example, a different microphone channel that is currently deactivated may now be closer to the new location of the DSM head, while the currently activated microphone channel may no longer be close to the DSM head. In such cases, the microphone channel that is not closest to the DSM head may be activated (e.g., turned ON), while the remaining microphone channels (including the previously activated microphone channel) may be deactivated (e.g., turned OFF). In some aspects, the updated distance information from the microphone device to the DSM head may also be used to update the gain and filter settings of the microphone device to obtain a clean and usable audio signal from the microscope head location where the surgeon is located. After block 210, the computing device may repeat steps 202 through 208 through 212 as part of its periodic monitoring of the position of the DSM head.

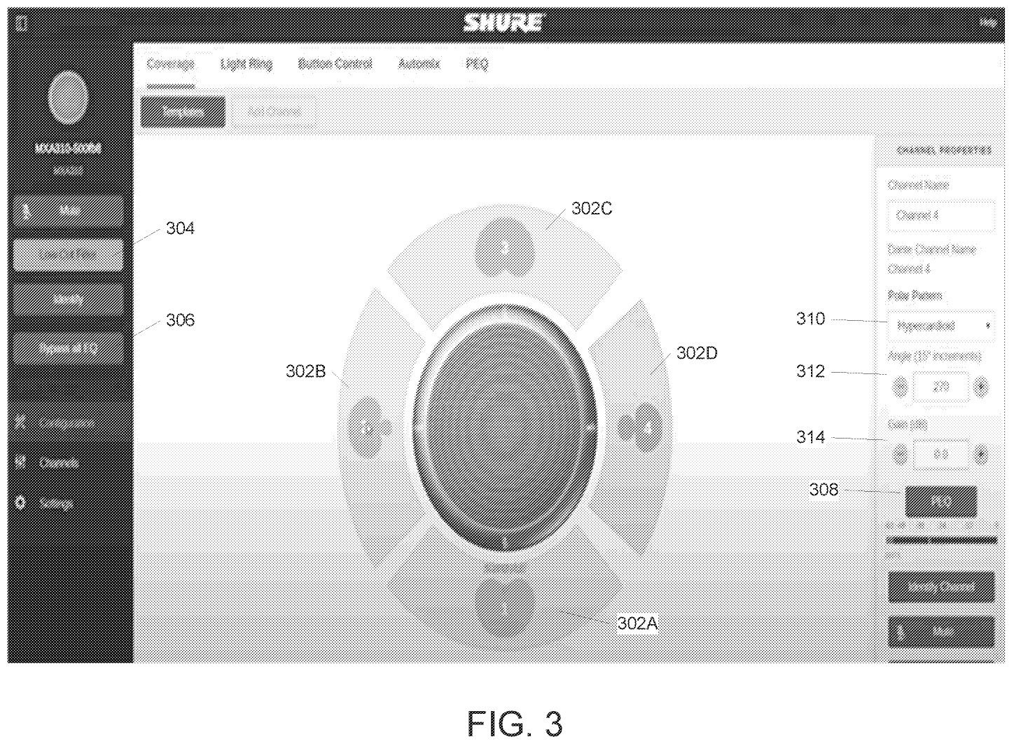

FIG. 3 shows a screenshot of a user interface associated with a system for controlling microphone directionality in a surgical environment, according to an example embodiment of the present disclosure. The user interface may be associated with a software application managed by a software API for a microphone device having a plurality of direction-based channels. The application may allow an operator or user of the surgical navigation and visualization system to see the microphone's directionality controlled based on a surgeon's command according to systems and methods presented herein. For example, the application may show that a single channel 302D may be activated that is directed to (e.g., located in the direction of) the user (e.g., surgeon). The remaining channels 302A-302C are shown as deactivated as their deactivation coupled with the activation of channel 302A may help to reduce background noise and false positives. The application may also provide the operator or user with additional controls to further optimize the microphone device. For example, the signal to noise (SNR) can be further increased by filtering (low pass and high pass combinations) the signal (e.g., “low-cut filter” 304) and modifying the in-built equalizers (e.g., via “bypass all EQ” 306, “PEQ” 308). The application may also allow the user to manually modify the polar pattern of the direction-based channels (e.g., currently set at “hypercardoid” 310), adjust the angle of the direction-based channels (e.g. “angle” 312), and adjust the gain 314. For example, the hypercardioid polar pattern may be useful in achieving the narrowest pickup range. Furthermore, in some aspects, the direction-based channels of the microphone device may be directed to a specific direction by rotating the channel around the disc. Although the application may provide manual control of various microphone device settings to the user, it is to be appreciated that one or more of these functionalities (e.g., gain, activation and/or deactivation of direction-based channels) may be set automatically based on the location of the DSM head as described in the present disclosure (e.g., in relation to FIG. 2).

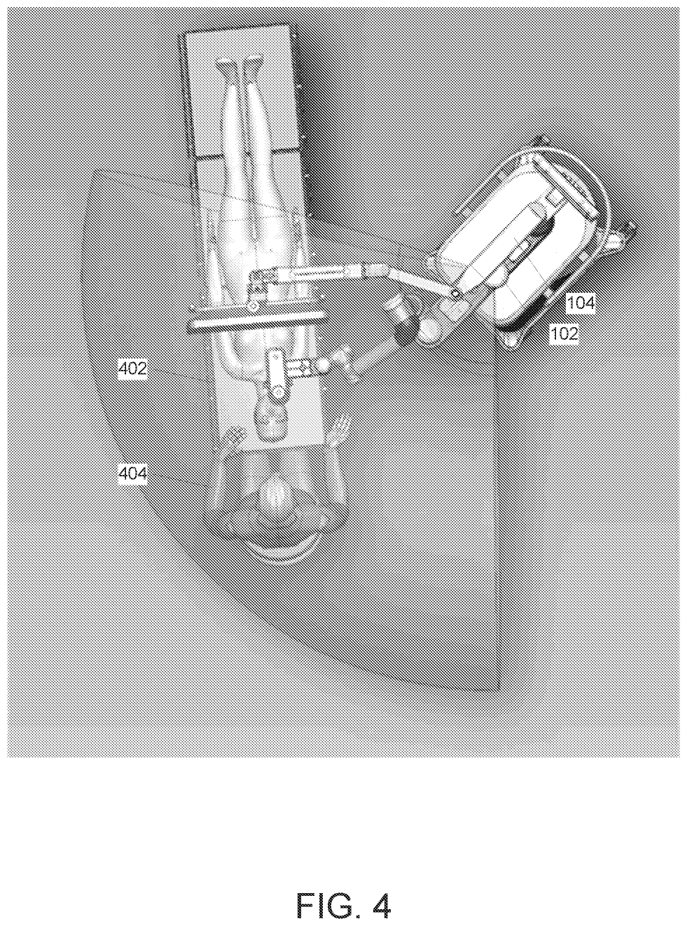

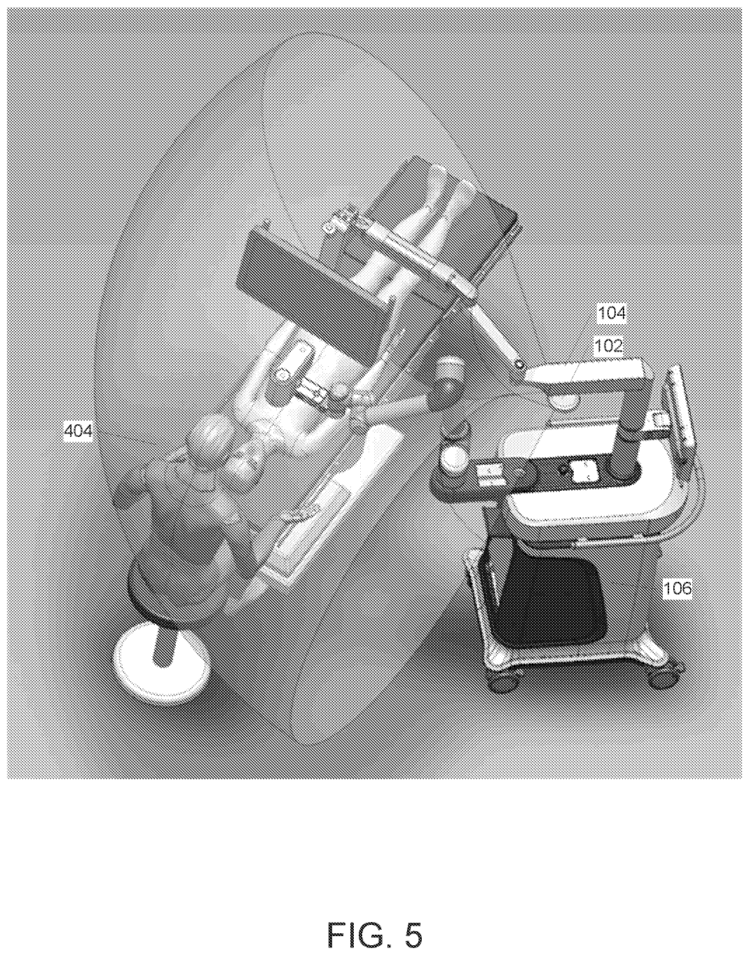

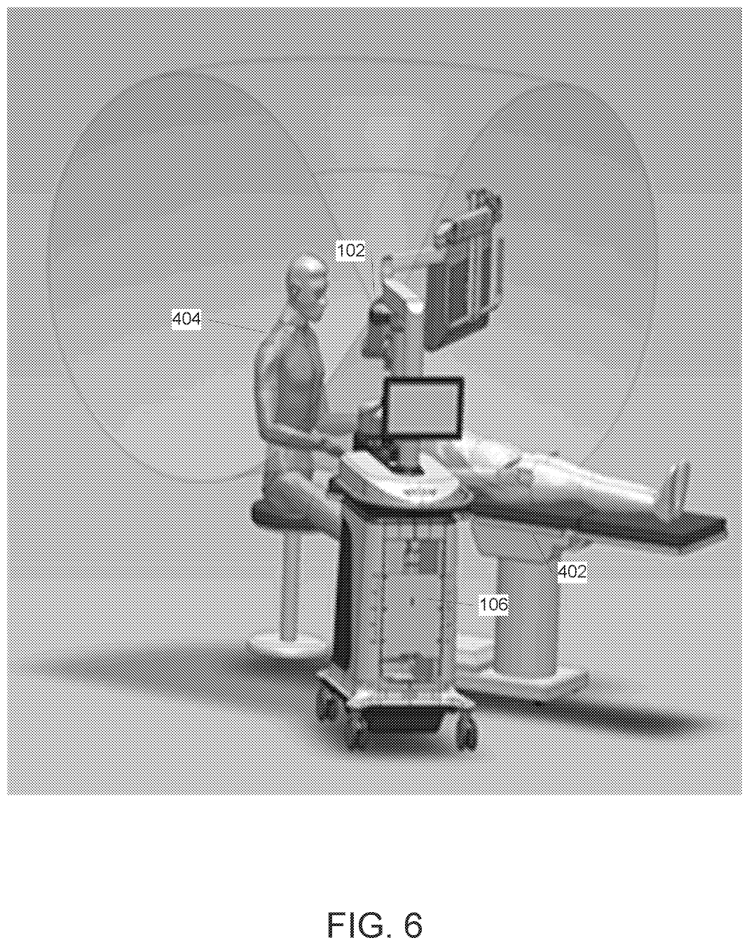

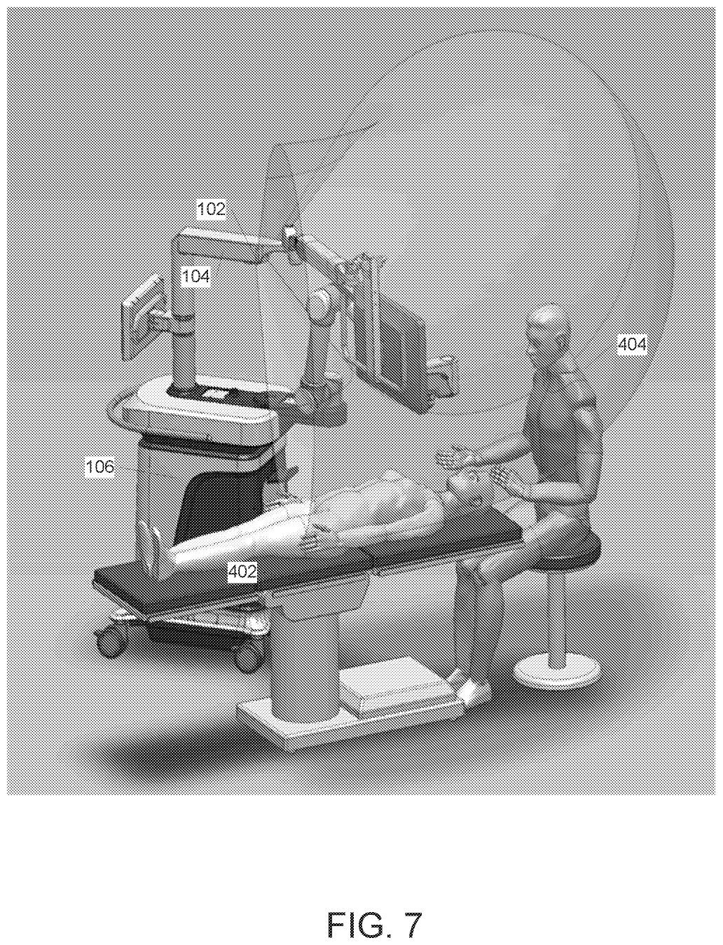

FIGS. 4-7 show illustrations, in different rotational positions, of a surgical environment for providing microphone directionality based on a surgeon's command, according to example embodiments of the present disclosure. In each of these illustrations, the microphone device 102 is located on the mast 108. Since the mast typically does not move during a surgical procedure, the mast 108 may hence be a stable location for the microphone device 102. In some aspects, the microphone device 102 may be placed upside down to avoid potential issues with liquid ingress. As shown in these illustrations, the cart 106 may be positioned close to the operating room table 402. Hence, it may be safe to assume that a surgeon 404 may be in the working volume of the microphone device 102. The operating room table 402 may provide an area for the patient to rest or otherwise be in a position for the surgical procedure.

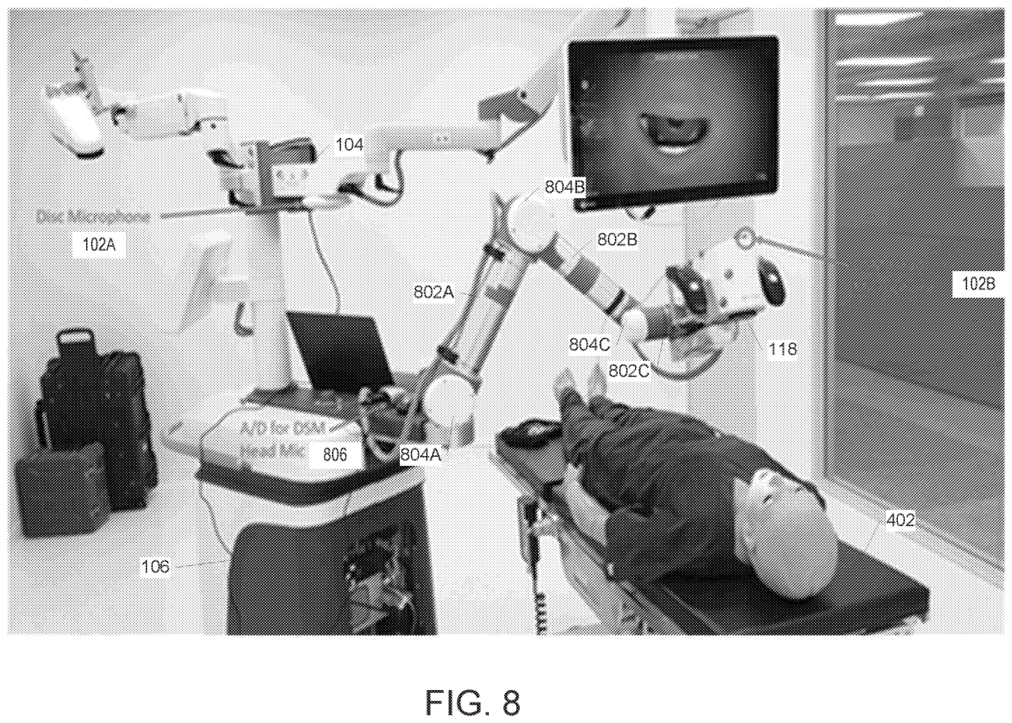

FIG. 8 is an illustration showing a surgical environment for providing microphone directionality based on a surgeon's command, in which a microphone device is placed in multiple locations to test its efficacy, in accordance with non-limiting embodiments of the present disclosure. As shown in FIG. 8, the surgical environment includes the mast 104, the cart 106, the operating room table 402, the DSM head 118, robotic arms 802A-802C leading to the DSM, and robotic joints 804A-804C providing movement for the robotic arms 802A-802C. These components may serve functions as previously described in relation to FIGS. 1, and 4-7.

Moreover, FIG. 8 shows different options for the placement of the microphone device 102. In one option, the microphone device is placed on the mast 104 (e.g., disc microphone 102A). The disc microphone device may be oriented horizontally or vertically. This location option, particularly a disc microphone device in a horizontal orientation, is favored for reasons discussed herein. In another option, the microphone device is placed at the head of the DSM (“DSM Head microphone” 102B). The DSM Head microphone 102B may communicate with an analog to digital converter (“A/D for DSM Head Mic” 806) placed near the cart, which may convert the analog signals received by the DSM Head microphone 102B into digital signals that may processed as executable commands. Also or alternatively, the location of A/D for DSM Head Mic 806 at the base of the cart 106 may be a third option for the placement of the microphone device. Each placement option may provide different results. A comparison of the efficacy of these microphone placement options will be discussed herein, in relation to subsequent figures.

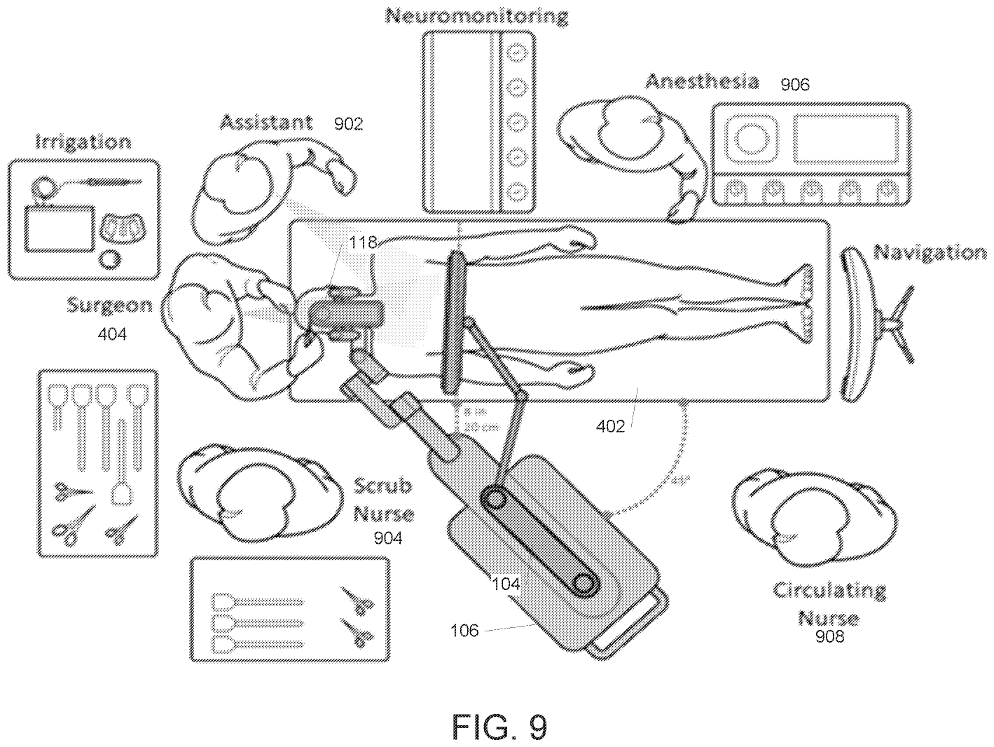

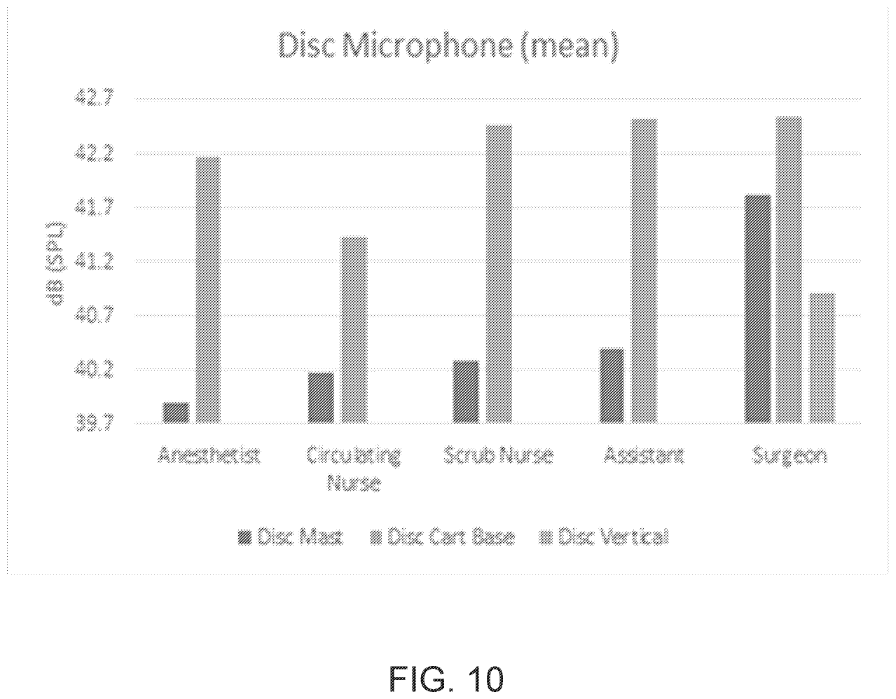

FIG. 9 is an illustration of the location of different users in a surgical environment for testing the optimal location of the microphone device, in accordance with a non-limiting embodiment of the present disclosure. As shown in FIG. 9, a surgeon 404 may typically be situated near the DSM head 118, since the surgeon 404 may need to attend to the surgical site throughout the procedure. A surgeon assistant 902 and a scrub nurse 904 may be on either side of the surgeon 404, not too far from the DSM head 118 but also not nearly as close as the surgeon 404. The anesthetist 906 and the circulating nurse 908 may be furthest from the DSM head 118, almost standing opposite from the surgeon 404. However, the circulating nurse may be closer to the mast 104, since the cart 106 on which the mast 104 is attached may be situated at the side of the operating table 402. As previously discussed, the microphone device may be located at the mast (e.g., Disc microphone 102A in FIG. 8) or on the DSM head (e.g., DSM Head microphone 102B in FIG. 8). However, as will be described in relation to FIGS. 10-15, experimental results of the quality of the voice commands received by each microphone device may show that the placement of the microphone device on the mast is most optimal for providing voice control to the surgeon.

For an assessment of the most optimal location of the microphone device (e.g., on the mast or on the DSM head), the output of the microphone device was recorded from sound coming from the different users shown in the operating room layout of FIG. 9. For example, for each microphone device location (on the mast and on the DSM head), the output was recorded for sound (e.g., voice) from each user at their respective location (e.g., surgeon 404, surgeon assistant 902, scrub nurse 904, anesthetist 906, circulating nurse 908). SPL (Sound Pressure Level) was calculated for the recorded signals and compared for analysis. In order to compare the sound quality of the microphone device at both locations (e.g., on the mast and on the DSM head), a low pass filter was used to reduce fan, HVAC, and other non-relevant background noise. The same set of basic voice commands, such as “Start Recording,” “Stop Recording,” “Zoom in,” “Zoom Out,” and “Focus,” were used by each user. The incoming sound signal at the microphone device was trimmed to remove preceding and trailing blank spaces that did not contain any commands. The resulting signal was processed in MATLAB using SPLMeasure.m script to obtain sound pressure values in decibels (dB). A mean and a max for the sound pressure data was calculated.

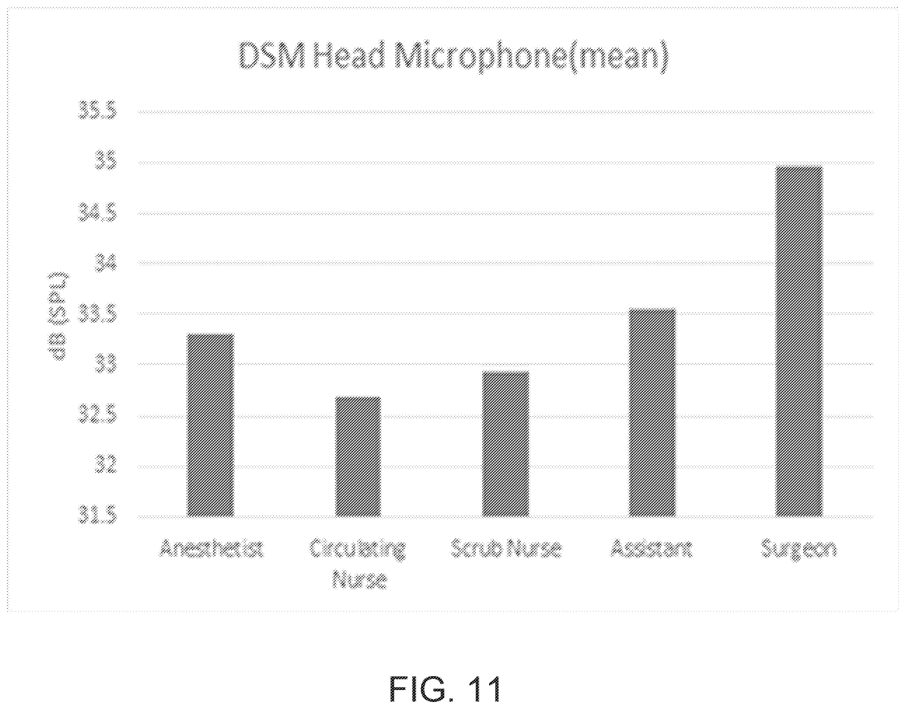

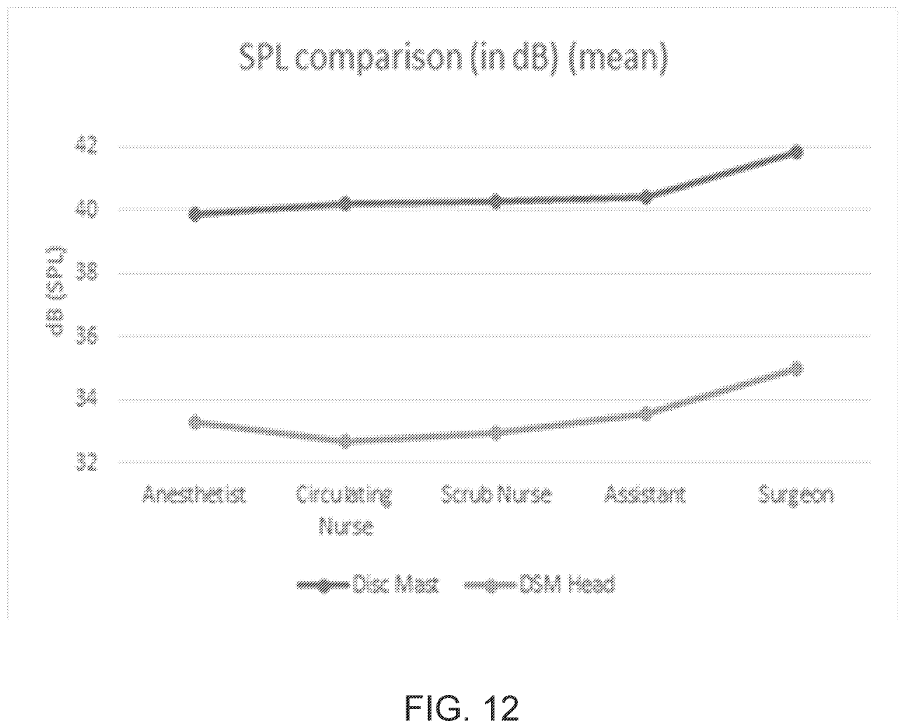

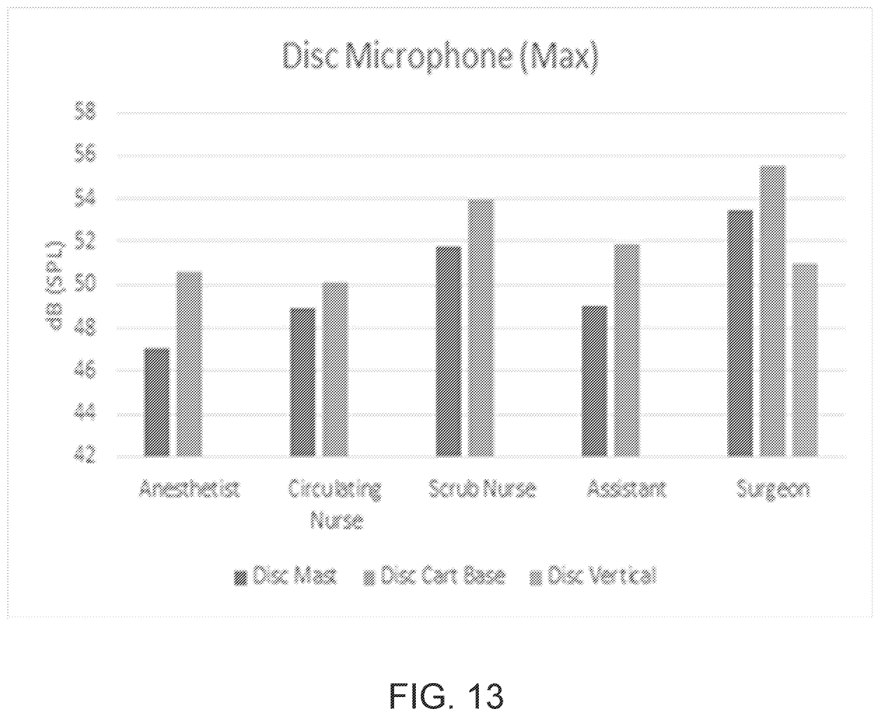

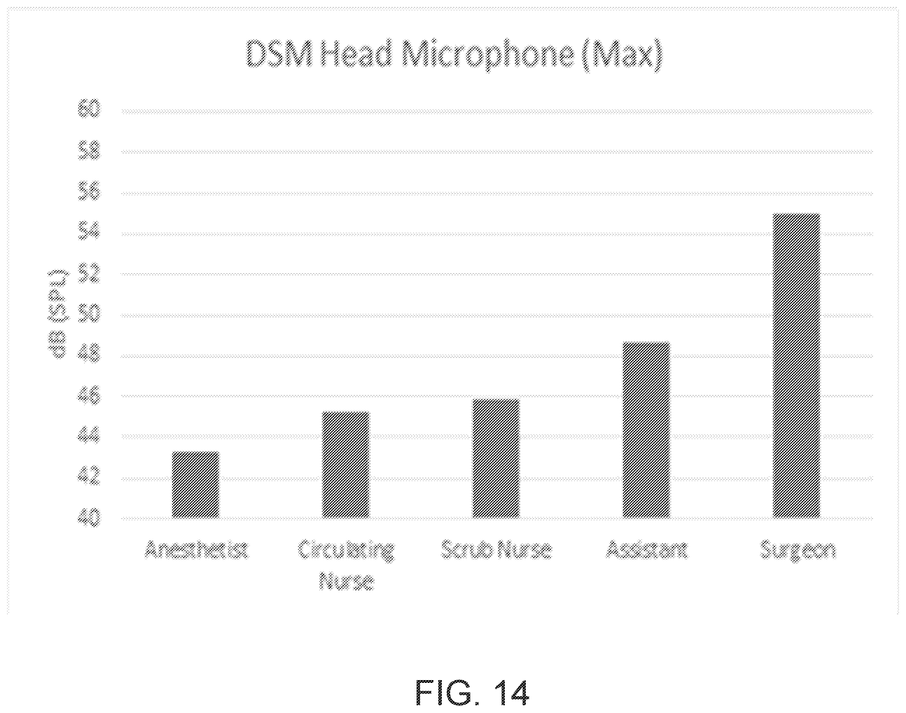

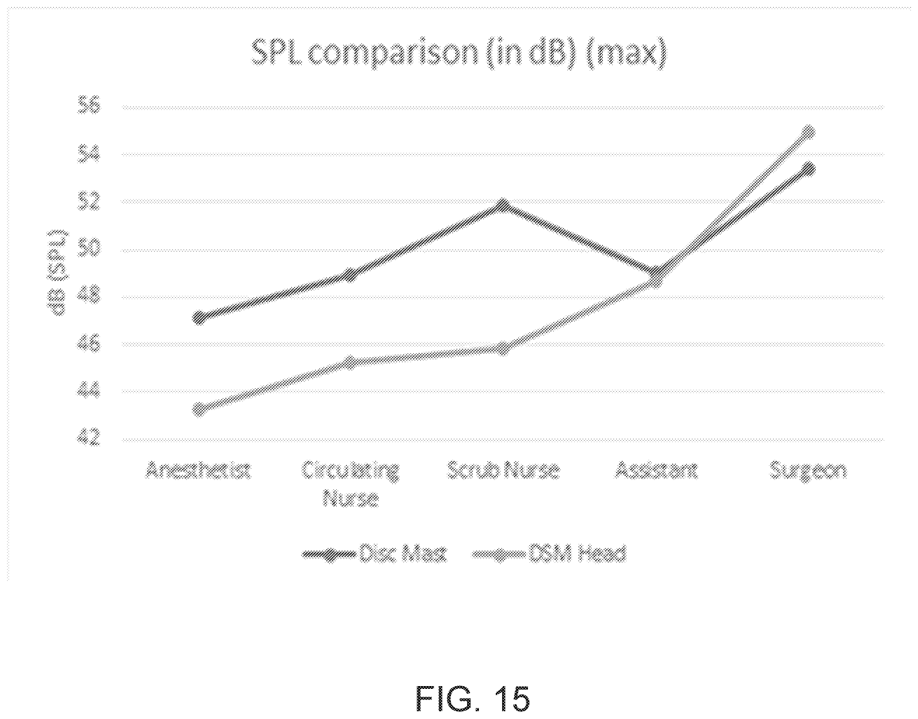

For example, while FIG. 10 is a graph of a mean sound analysis of a disc microphone device placed in different locations (e.g., on the mast, on the base of the cart 106), and orientations (e.g. vertical), whereas FIG. 13 is a graph of the max sound analysis of the disc microphone placed in those same locations and orientations. Furthermore, while FIG. 11 is a graph of a mean sound analysis of a microphone device placed at the DSM head, FIG. 14 is a graph of a max sound analysis of that microphone device placed at the DSM head. A comparison of the mean sound analysis of the disc microphone device placed on the mast with the mean sound analysis of a microphone device placed at the DSM head is shown in FIG. 12, whereas a comparison of the max sound analysis of the disc microphone device placed on the mast with the max sound analysis of the microphone device placed at the DSM head is shown in FIG. 15. For each of these graphs, the measured sound pressure is based on voice commands provided by the anesthetist, circulating nurse, scrub nurse, assistant and surgeon (e.g., see X-axis of FIGS. 10-15).

The results show that the disc microphone may be sensitive to the direction it is pointed towards. For example, as shown in FIGS. 10 and 13, the disc microphone placed in a vertical orientation does not register as high of a sound pressure from the surgeon as a disc microphone placed horizontally (e.g., on the mast or on the cart base). Thus, the vertical orientation of a disc microphone reduces the output if user talks from surgeon's position. The results also show that the disc microphone (e.g., placed on the mast or the cart base) may have a higher output at 0 dB gain as compared to DSM head mic at 50% increased gain. The microphone output was comparable for the surgeon, assistant and the scrub nurse when the disc microphone is placed on the cart base.

Furthermore, the results show that the microphone device placed on the DSM head may have a shorter range and hence may be more sensitive to surgeon's voice commands. However, since a microphone device placed on the DSM head may be prone to movements caused by the movement of the DSM head during the course of the surgery, a microphone device on the mast may be more stable and unaffected from changes. Furthermore, the methods of activating direction-based channels based on a determination of the location of the DSM head, as previously discussed herein, may allow the microphone device located on the mast to be more sensitive to a surgeon's voice commands.

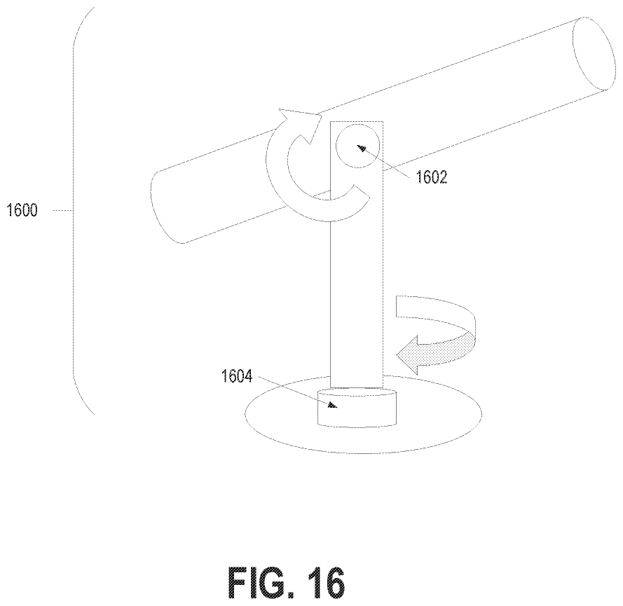

In some embodiments, microphones other than disc microphones may utilize the systems and methods described above for optimizing microphone directionality. For example, FIG. 16 shows a traditional (e.g., shotgun) microphone 1600 mounted on motorized turrets 1602 and 1604 implementing the systems and methods for providing microphone directionality based on surgeon command, in accordance with a non-limiting embodiment of the present disclosure. As shown in FIG. 16, motorized turret 1602 allows the microphone 1600 to rotate so as to move the microphone from pointing up to pointing down, while motorized turret 1604 allows the microphone 1600 to rotate so as to move the microphone so that sound in any of the cardinal directions can be received. Although the microphone 1600 shown in FIG. 16 has two motorized turrets, it is contemplated that microphones may have any number of joints for movement and/or rotation, motorized or unmotorized. The directionality for these microphones may be controlled via the same manner as the existing disk microphone, for example by rotating the microphone along axes of the motorized turrets. In some aspects, the “directionality” of such microphones may be determined based on how the microphone is positioned along the axes of each of the joints, for example, the joints having motorized turrets 1602 and 1604. In some embodiments, the one or more processors 124 of the computing device 122 and/or of the microphone device 1600 may determine the location of the DSM head 118 respective to the microphone 1600. For example, as previously discussed, the location may be determined from received joint angle information of the robotic arms holding the DSM head 118. The one or more processors 124 may cause the motorized turrets 1602 and 1604 to move (e.g., by rotating) the microphone head towards a direction that is closest to the determined location of the DSM head 118. By appropriately positioning the microphone 1600 toward the DSM head 118, the microphone 1600 may thus be more receptive to the surgeon's commands, since the surgeon is likely to be near the DSM head 118.

The optimized directionality of the various microphones described herein may result in the microphones becoming more effective at receiving and facilitating the execution of voice commands by surgeon. For example, by activating those channels of the microphone that are closest to the location of the DSM head 118 (and therefore to the surgeon), and deactivating the channels that are farthest from the DSM head 118, microphones may reduce or eliminate noise that affects the processing of voice commands. Similarly, by causing the rotation and/or movement of a traditional microphone (e.g., via motorized turrets) towards the location of the DSM head 118, the traditional microphone may be better able to receive the voice commands of the surgeon, while minimizing background noise. The voice commands may be received by the microphone as sound signals, converted to digital signals, and recognized as various commands for the computing device 122 to execute. For example, the microphone device may transmit (e.g., via the output module) a digital signal corresponding to the voice command to the computing device 122. The computing device 122 may identify, based on the digital signal, the voice command as a microscope movement command. The computing device 122 may cause the digital surgical microscope to execute the microscope movement command, for example, by sending electrical signals to actuators of the robotic joints responsible for moving the DSM head 118. Non-limiting examples of microscope movement commands include but are not limited to: an X-Y movement command along a field of view of the DSM head 118 (e.g., shifting the field of view in a Cartesian direction); a lock-to-target command within the field of view (e.g., to ensure that the field of view DSM head does not shift away from a target region); a Z-axis movement command towards or away from the field of view (e.g., for focus); or a yaw movement command.

Also or alternatively, the computing device 122 may identify, based on the digital signal, the voice command as a non-movement control. For example, the computing device 122 may identify a focus command, an autofocus command (e.g., to cause the DSM head 118 to automatically default to a desired or predetermined focus setting), a zoom command, a white light intensity command, or a toggle (e.g., ON/OFF) command. In some embodiments the voice commands may pertain to the capture, adjustment, or use of certain media by the DSM head 118 (e.g., a snapshot command (e.g., to capture an image), an image and/or color adjustment command; an image scrolling command (e.g., to go to a previous or next image); a recording command (e.g., to capture a video); a bookmark command (e.g., to save an image or video); or a hyperspectral imaging command (e.g., to capture or adjust a hyperspectral image or video). In some aspects, the hyperspectral imaging commands may include functionalities for near infrared imaging (NIR), near ultraviolet imaging (NUV), and/or fluorescence imaging. For example, hyperspectral imaging commands may include but are not limited to: a toggle command for NIR imaging (e.g., NIR at the 800 nanometer visible region (NIR 800)); a playback command for the NIR imaging; a light control command for the NTR imaging; a toggle command for near ultraviolent imaging (e.g., at the 400 nanometer visible region (NUV 400)); a playback command for the NUV imaging; a light control command for the NUV imaging; a toggle command for fluorescence imaging; a playback command for the fluorescence imaging; a light control command for the fluorescence imaging; a toggle command for processing-in-pixel (PIP); or a swap sources command (e.g., to switch between different hyperspectral imaging options). The computing device 122 may thus cause the digital surgical microscope to execute the identified voice command.

### CONCLUSION

It will be appreciated that each of the systems, structures, methods and procedures described herein may be implemented using one or more computer programs or components. These programs and components may be provided as a series of computer instructions on any conventional computer-readable medium, including random access memory (“RAM”), read only memory (“ROM”), flash memory, magnetic or optical disks, optical memory, or other storage media, and combinations and derivatives thereof. The instructions may be configured to be executed by a processor, which when executing the series of computer instructions performs or facilitates the performance of all or part of the disclosed methods and procedures.

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims. Moreover, consistent with current U.S. law, it should be appreciated that 35 U.S.C. 112(f) or pre-AIA 35 U.S.C. 112, paragraph 6 is not intended to be invoked unless the terms “means” or “step” are explicitly recited in the claims. Accordingly, the claims are not meant to be limited to the corresponding structure, material, or actions described in the specification or equivalents thereof.

What is claimed is:

1. A system comprising:
a movable cart;
a mast affixed to the movable cart;
one or more robotic arms, each robotic arm associated with one or more rotating elements, each rotating element associated with a respective sensor to detect angle information;
a coupler affixed to a distal end of a robotic arm of the one or more robotic arms
a digital surgical microscope affixed to the coupler;
a microphone device affixed to the mast, wherein the microphone device comprises a plurality of channels associated with a plurality of directions;
a processor; and
memory storing computer-executable instructions that, when executed by the processor, causes the system to:
receive, via the respective sensor for each of the one or more rotating elements, an angle information for each rotating element;
determine, based on the angle information for each rotating element, a joint angle information for the digital surgical microscope;
determine, based on the joint angle information, a location of a head of the digital surgical microscope respective to the microphone device; and
activate, based on the location, a first channel of the plurality of channels associated with the plurality of directions, wherein first channel is associated with a first direction of the plurality of directions indicative of the location.

2. The system of claim 1, wherein the computer-executable instructions, when executed, further cause the system to:
deactivate, based on the location, remaining channels of the plurality of channels associated with the plurality of directions.

3. The system of claim 1, wherein the computer-executable instructions, when executed, cause the system to determine the location of the head of the digital surgical microscope by:
determining, upon an initialization of the system, an initial location of the microscope;
activating, based on the initial location, an initial channel of the plurality of channels; and
generating, based on a comparison of the initial location to the location of the microscope, a distance vector of the microscope.

4. The system of claim 3, wherein the computer-executable instructions, when executed, cause the system to determine the location of the head of the digital surgical microscope by:
prior to activating the first channel,
determining that the distance vector of the microscope satisfies a predetermined threshold; and
deactivating the initial channel of the plurality of channels, wherein the first channel is different from the initial channel.

5. The system of claim 3, wherein the computer-executable instructions, when executed, cause the system to determine the location of the head of the digital surgical microscope by:
determining that the distance vector of the microscope does not satisfy a predetermined threshold,
wherein the initial channel comprises the first channel,
wherein the activating the first channel comprises maintaining the activation of the initial channel.

6. The system of claim 1, wherein the computer-executable instructions, when executed, further cause the system to:
determine, based on the location of the head of the digital surgical microscope, a proximity of the digital surgical microscope to the microphone device; and
adjusting, based on the proximity, a gain of the microphone device.

7. The system of claim 1, wherein the computer-executable instructions, when executed, further cause the system to:
filter, via a low pass and high pass filter, an audio signal received from the first channel to increase a signal to noise ratio (SNR); and
modify one or more equalizers of the microphone device to isolate a voice command associated with the audio signal.

8. The system of claim 1, wherein the microphone device further comprises an output module for converting voice commands filtered through one or more of the plurality of channels into digital signals.

9. The system of claim 8, wherein the computer-executable instructions, when executed, further cause the system to:
receive, via the output module of the microphone device, a digital signal corresponding to a voice command;
identifying, based on the digital signal, the voice command as a microscope movement command; and
causing the digital surgical microscope to execute the microscope movement command.

10. The system of claim 9, wherein the microscope movement command comprises one or more of:
an X-Y movement command along a field of view;
a lock-to-target command within the field of view;
a Z-axis movement command towards or away from the field of view; or
a yaw movement command.

11. The system of claim 8, wherein the computer-executable instructions, when executed, further cause the system to:
receive, via the output module of the microphone device, a digital signal corresponding to a voice command;
identifying, based on the digital signal, the voice command as one of:
a focus command;
an autofocus command;
a zoom command;
a white light intensity command;
a toggle command;
a snapshot command;
an image and/or color adjustment command;
an image scrolling command;
a recording command;
a bookmark command; or
a hyperspectral imaging command; and
causing the digital surgical microscope to execute the voice command.

12. The system of claim 8, wherein the hyperspectral imaging command is one or more of:
a toggle command for near infrared imaging;
a playback command for the NIR imaging;
a light control command for the NIR imaging;
a toggle command for near ultraviolent (NUV) imaging;
a playback command for the NUV imaging;
a light control command for the NUV imaging;
a toggle command for fluorescence imaging;
a playback command for the fluorescence imaging;
a light control command for the fluorescence imaging;
a toggle command for processing-in-pixel (PIP); or
a swap sources command.

13. A method of controlling microphone directionality in a surgical environment, the method comprising:
receiving, by a computing device having a processor, via a respective sensor for each of one or more rotating elements of each of one or more robotic arms connecting a digital surgical microscope to the computing device, an angle information for each rotating element;
determining, by the computing device, based on the angle information for each rotating element, a joint angle information for the digital surgical microscope;
determining, based on the joint angle information, a location of a head of the digital surgical microscope respective to a microphone device; and
activating, based on the location, a first channel of a plurality of channels of the microphone device.

14. The method of claim 13, wherein the plurality of channels of the microphone device are associated with a respective plurality of directions, wherein the first channel is associated with a first direction of the plurality of directions.

15. The method of claim 13, further comprising:
deactivating, based on the location and responsive to the activation of the first channel, remaining channels of the plurality of channels of the microphone device.

16. The method of claim 13, further comprising:
determining, upon an initialization of the system, an initial location of the microscope;
activating, based on the initial location, an initial channel of the plurality of channels; and
generating, based on a comparison of the initial location to the location of the microscope, a distance vector of the microscope.

17. The method of claim 16, wherein determining the location of the head of the digital surgical microscope comprises:
prior to activating the first channel,
determining that the distance vector of the microscope satisfies a predetermined threshold; and
deactivating the initial channel of the plurality of channels, wherein the first channel is different from the initial channel.

18. The method of claim 16, wherein determining the location of the head of the digital surgical microscope comprises:
determining that the distance vector of the microscope does not satisfy a predetermined threshold,
wherein the initial channel comprises the first channel,
wherein the activating the first channel comprises maintaining the activation of the initial channel.

19. The method of claim 13, further comprising:
determining, based on the location of the head of the digital surgical microscope, a proximity of the digital surgical microscope to the microphone device; and
adjusting, based on the proximity, a gain of the microphone device.

20. The method of claim 13, further comprising:
filtering, via a low pass and high pass filter, an audio signal received from the first channel to increase a signal to noise ratio (SNR); and
modifying one or more equalizers of the microphone device to isolate a voice command associated with the audio signal.

21. The method of claim 13, further comprising:
receiving, via the microphone device, a digital signal corresponding to a voice command;
identifying, based on the digital signal, the voice command as a microscope movement command; and
causing the digital surgical microscope to execute the microscope movement command.

22. The method of claim 21, wherein the microscope movement command comprises one or more of:
an X-Y movement command along a field of view;
a lock-to-target command within the field of view;
a Z-axis movement command towards or away from the field of view; or
a yaw movement command.

23. The method of claim 13, further comprising:
receiving, via the microphone device, a digital signal corresponding to a voice command;
identifying, based on the digital signal, the voice command as one of:
a focus command;
an autofocus command;
a zoom command;
a white light intensity command;
a toggle command;
a snapshot command;
an image and/or color adjustment command;
an image scrolling command;
a recording command;
a bookmark command; or
a hyperspectral imaging command; and
causing the digital surgical microscope to execute the voice command.

24. The method of claim 23, wherein the hyperspectral imaging command is one or more of:
a toggle command for near infrared imaging;
a playback command for the NIR imaging;
a light control command for the NIR imaging;
a toggle command for near ultraviolent (NUV) imaging;
a playback command for the NUV imaging;
a light control command for the NUV imaging;
a toggle command for fluorescence imaging;
a playback command for the fluorescence imaging;
a light control command for the fluorescence imaging;
a toggle command for processing-in-pixel (PIP); or
a swap sources command.

25. A non-transitory computer readable medium storing instructions that, when executed by a processor, cause the processor to perform steps comprising:
receiving, from a respective sensor for each of one or more rotating elements of each of one or more robotic arms connecting a digital surgical microscope to the computing device, an angle information for each rotating element;
determining, based on the angle information for each rotating element, a joint angle information for the digital surgical microscope;
determining, based on the joint angle information, a location of a head of the digital surgical microscope respective to a microphone device;
activating, based on the location, a first channel of a plurality of channels of the microphone device, wherein the plurality of channels of the microphone device are associated with a respective plurality of directions, wherein the first channel is associated with a first direction of the plurality of directions; and
deactivating, based on the location, remaining channels of the plurality of channels of the microphone device.

26. The non-transitory computer readable medium of claim 25, wherein the instructions, when executed by the processor, further cause the processor to perform steps comprising:
receiving, via the microphone device, a digital signal corresponding to a voice command;
identifying, based on the digital signal, the voice command as a microscope movement command; and
causing the digital surgical microscope to execute the microscope movement command.
